(19) United States
(12) Patent Application Publication
(10) Pub. No.: US 2016/0243460 A1
(43) Pub. Date: Aug. 25, 2016

(54) Thermal Fractionation Of Plant Material

(71) Applicant: Biofract, LLC, Potsdam, NY (US)

(72) Inventors: Philip D. Leveson, Sandy Springs, GA (US); John Paul Gaus, Watertown, NY (US)

(21) Appl. No.: 15/046,825

(22) Filed: Feb. 18, 2016

Related U.S. Application Data
(60) Provisional application No. 62/117,981, filed on Feb. 19, 2015.

(51) Int. Cl. B01D 3/14; B01D 11/02; A61K 36/82; A61K 31/015; A61K 31/575; A61K 31/122; A61K 31/01; A61K 31/085; A61K 31/353; A61K 31/045; A61K 31/05; B01D 3/02; A61K 31/352

(52) U.S. Cl. CPC B01D 3/143; B01D 3/02; B01D 11/0215; A61K 36/82; A61K 31/015; A61K 31/575; A61K 31/352; A61K 31/01; A61K 31/085; A61K 31/353; A61K 31/045; A61K 31/05; A61K 31/122; B01D 2011/007

(57) ABSTRACT

A highly controllable thermal distillation reactor system (25) and method (30) allow volatile compounds to be efficiently removed from the plant matter without use of solvents, and can separate compounds with different vapor pressure characteristics. The system has a Thermal Distillation Reactor (TDR) (13) into which the plant material is charged. The TDR provides for high rates of heat transfer coupled with small thermal diffusion length scales to allow substantially all of the plant material to be within a narrow temperature range, which enhances the separation purity cuts. The system provides high removal efficiencies whilst minimizing any impurities resulting from pyrolysis or thermal destruction to the cellulose, hemi-cellulose or lignin within the plant material.

### CROSS-REFERENCE TO RELATED APPLICATION

This application claims the priority of U.S. Provisional Patent Application No. 62/117,981, filed Feb. 19, 2015, entitled “Thermal Fractionation of Plant Material,” the entire disclosure and contents of which are incorporated herein by reference.

### BACKGROUND OF THE INVENTION

Many plant species contain chemical compounds which hold medicinal and/or therapeutic properties. The active components are often in the form of a complex mixture of active components, analogues and isomers. The synergistic enhancements of these active compounds to vitality are not fully understood and the optimum ratio of the different compounds or groups is not known. It is believed, however, that plant based treatments for health may be optimized by controlling the ratios of these active compounds to maximize synergistic health benefits.

Many plant species contain chemical compounds which hold medicinal and/or therapeutic properties. These compounds are often separated from the bulk plant material using solvent based chemical extraction processes. Utilizing solvents has a number of drawbacks. The solvent typically has poor selectivity to the target range of chemicals compounds and can remove chlorophyll, waxes and other unwanted plant components. The solvent extraction process is non-specific and may extract some of the active compounds preferentially depending upon the polarity of both the solvent and the active compound. After the active compounds have been extracted from the plant material the two phase (solid, liquid) mixture is filtered to separate any solids from the liquid phase. The liquid phase is then typically heated to evaporate the solvent to leave the active product. This approach has a number of drawbacks. Even after a long period of evaporation some residual solvent will typically remain in the residual liquid or phase. Often, the long term effect of these solvents on human health is unknown. In a worst case scenario these solvents can be problematic to human health, cause inflammations and rashes if administered via skin contact, and/or may even be known to be carcinogenic. Due to the effects of residual solvents on human health a safer solvent, but with less favorable extraction properties, is often used. A poor solvent can result, however, in a product of very low purity and with non-ideal variations of selectivity to active compounds within the plant matter. The use of solvents can also result in noxious and undesired emissions into the air, water, and/or ground, and can also result in explosions unless the solvents are treated with the upmost caution.

### BRIEF SUMMARY OF THE INVENTION

An apparatus thermally fractionates and recovers compounds contained within plant material. The apparatus includes a sweeper gas preheater, a distribution manifold, a plurality of channels connected to the distribution manifold, to receive macerated plant material, and to extract a gaseous product from the macerated plant material, a product collection manifold, a product recovery system, a heating device to heat either a liquid or a gas to a predetermined temperature, the plurality of channels being bathed in and heated by the liquid or the gas from the heating device, and an insulated container enclosing the distribution manifold, the plurality of channels, the collection manifold, and the product recovery system.

A method thermally fractionates and recovers compounds from plant based material. The method includes loading macerated plant material into a plurality of channels, introducing a sweeper gas flow along the length of each channel, externally heating each channel in accordance with a temperature controlled ramp, directing gas exiting the channels in a first temperature range to a first condenser, and directing the gas exiting the channels in a second temperature range to a second condenser. The first condenser provides a first compound and the second condenser provides a second compound.

### BRIEF DESCRIPTION OF THE DRAWINGS

FIG. 1 is a schematic diagram of a Thermal Distillation Reactor (TDR).

FIG. 2 is a schematic of an extraction system using the TDR.

FIG. 3 is a flow chart illustrating the steps of the extraction process.

FIG. 4 shows an exemplary computer architecture suitable for performing the processes and functions described herein.

### DETAILED DESCRIPTION OF THE INVENTION

The process and equipment described herein allows for the removal of active compounds from plant material and allows the fractionation of these compounds. The active compounds can then be recombined in a ratio optimal for medical treatments.

A highly controllable thermal distillation reactor system allows volatile compounds to be efficiently removed from the plant matter and offers the opportunity to separate different compounds within the plant material due to differences in vapor pressure characteristics. The system preferably utilizes high rates of heat transfer coupled with small thermal diffusion length scales to allow the material in the reactor to be within a narrow temperature range so as to enhance the separation purity. The system also provides for high removal efficiencies whilst minimizing any impurities resulting from pyrolysis or thermal destruction of the desired active components, cellulose, hemi-cellulose, or lignin within the plant material.

Removing the active components from the plant material through a thermal process offers the potential of selectively removing volatile components from the inert plant matter. This process is carefully controlled to avoid thermal gradients from occurring within the bed. If thermal gradients are present then removal of the active component will occur at different rates through the bed, which may result in slow removal rates, reduced removal efficiencies, and/or possibly the local pyrolysis of some of the cellulosic material. Through careful selection of heating rates and hold periods at certain key temperature settings a number of compositions (also known as “cuts”) can be produced. The chemical concentration of the active components within each cut may be very different. The cuts produced at lower temperatures are rich in the more volatile active components whilst the cuts produced at higher temperatures are rich in the less volatile active compounds.

The various cuts may then be recombined in differing ratios to produce extracts which contain active components in ratios which differ from the ratios of these compounds found in the original plant matter. This allows medical treatments to be optimized by combining extracts having different ratios to yield a product which is optimized for the treatment of particular ailments for each individual patient. Recombining these different extracts in different proportions provides for the production of treatment compositions which can be chemically identified and reproduced, even when variations of active compound ratios differ from batch to batch of the starting plant matter.

The use of a highly controllable thermal distillation reactor system (25) allows volatile compounds to be efficiently removed from the plant matter and offers the opportunity to separate compounds with different vapor pressure characteristics. In a preferred implementation the reactor system comprises a heating system, a thermal distillation reactor, a purge gas system, a condenser collector, and a fractionation header system. To enable the thermal separation of different active compounds from within the plant material all of the material being processed is slowly heated in a very consistent manner to minimize or prevent thermal gradients. This may be, and is preferably, achieved through the use of a Thermal Distillation Reactor (TDR).

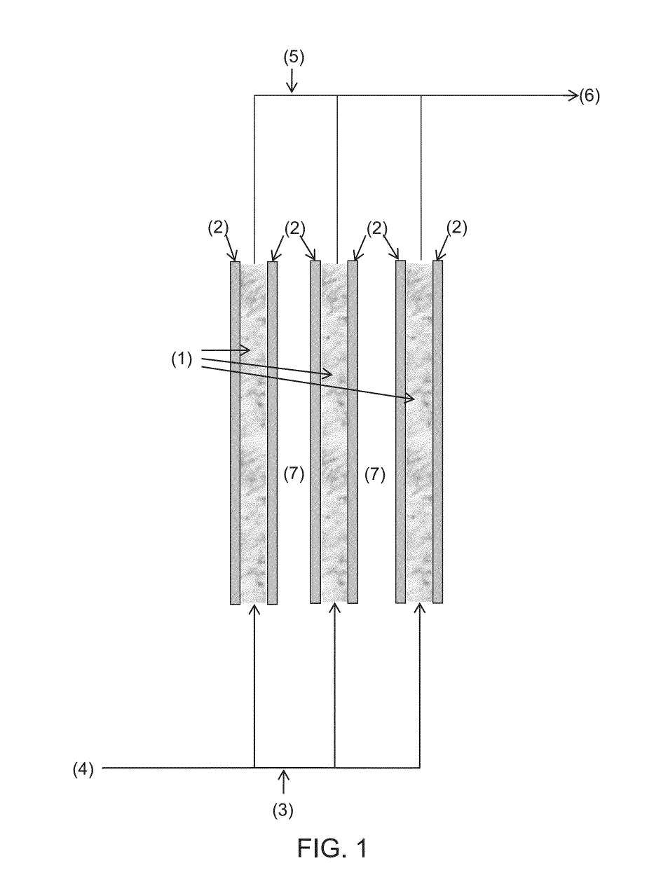

FIG. 1 is a schematic diagram of a TDR (13). The reactor consists of a number of distinct parallel channels (1) into which the macerated plant material is charged. The channels may be orientated in a vertical, horizontal or other desired or convenient position. The channels can be of any cross sectional geometry and area but preferably have a characteristic heat transfer dimension in the range 0.15″ to 6″. Examples of such channels include the inner volume of a pipe or an inner volume contained between two sealed plates. A tube or plate heat exchanger may be utilized. It is also possible to produce such an arrangement through the use of trays onto which the plant material is placed with a thickness in the preferred range 0.15″ to 6″. The tray may be solid or perforated, and may include a solid or perforated top piece. The plant material is initially macerated to produce particles with an average diameter or equivalent diameter in the preferred range of ⅕th to 1/100th that of the channel diameter into which the material is to be charged. The material is inserted into the channel (1) and may be vibrated to allow the material to uniformly settle within the channel. The material is preferably not tightly packed or packed into a solid form within the channel as this would result in a high pressure drop and promote the potential of a non uniform flow of sweeper gas through the channel. The walls of the channel enclosure (2) can be produced from any suitable heat resistant material, but stainless steel is preferred. A sweeper gas (4) is supplied to each parallel channel via an intake or distribution manifold system (3). The role of the sweeper gas (4) is to remove any vaporized compounds from the channel (1) as the plant material is heated. The sweeper gas (4) flows along the channel (1) and removes the vaporized active compounds via an exit (6) which is connected to each channel via a common exit manifold (5). A heat transfer fluid is arranged to flow through a path produced in the voids (7) between the process channels (2). A number of heat transfer fluids can be utilized, including but not limited to pressurized water, steam, hot oil and commercial heat transfer fluids. The channel containing the plant material may also be heated, alternatively or additionally, by using an electrical heating element. The heat transfer fluid may flow in a counter current flow, co-current flow, or crossflow, or be a well mixed random turbulent flow. The heat transfer fluid velocity is chosen such that the outlet temperature of the fluid is only slightly cooler, preferably less than 1° C. cooler, than the inlet temperature so as to minimize any thermal gradients within the packed channel.

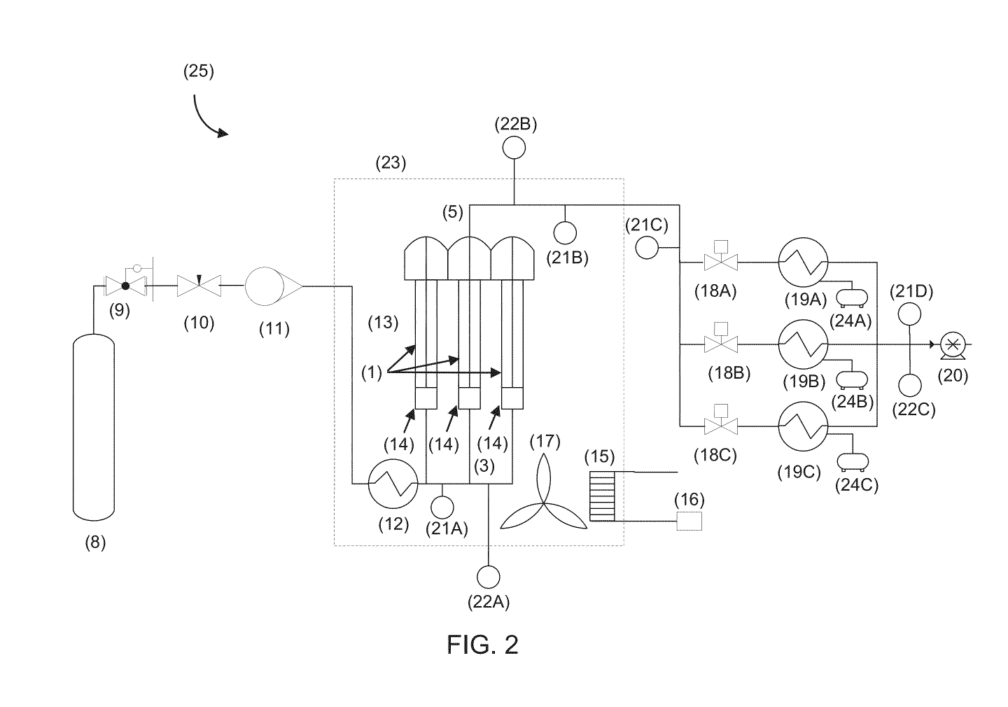

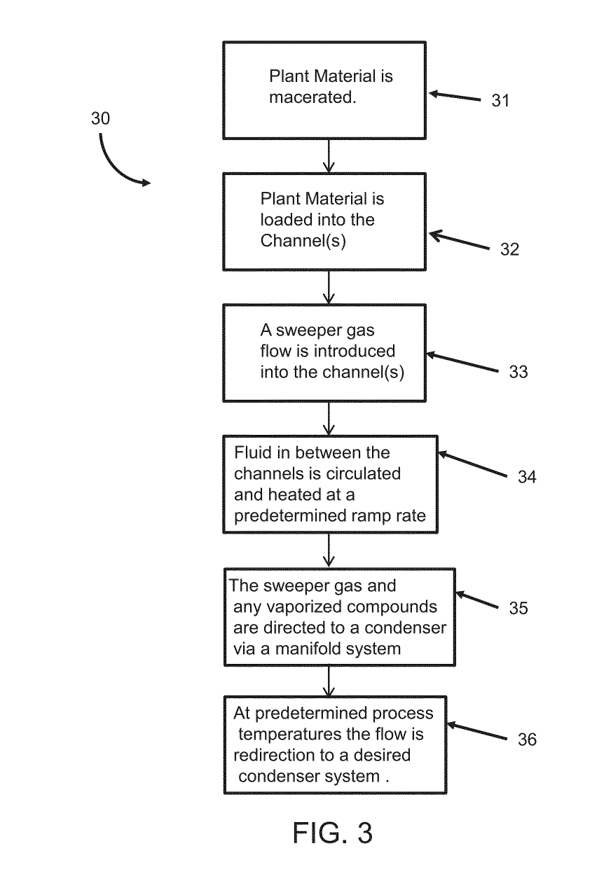

A process to allow for the thermal distillation of plant material preferably uses the TDR (13) in conjunction with other equipment or components. FIG. 2 is a schematic of an extraction or distillation system (25) using the TDR (13). FIG. 3 is a flow chart illustrating the steps of the extraction process (30). The plant material to be processed is first macerated (31) into a 0.015 to 1.2″ size range. The biomass is preferably at least partially pre-dried, but wet, green material may also be used directly. The whole plant, including root systems, branched, leaved and stems may be mixed and macerated, or selected elements of the plant may be mixed and macerated. The TDR (13) is then charged with the macerated, preferably at least partially dried, plant material. The material is charged (32) into the channel (1) and may be vibrated to allow the material to uniformly settle with the channel. The vibration intensity and duration, and the charging pressure, preferably do not pack the macerated material into a solid form within the channel.

Sweeper gas is supplied (33) to the process from a storage bottle (8) or from a pipeline of any other gas delivery device. The sweeper gas can be any inert or substantially inert gas, examples of such include but are not limited to permanent gases (e.g. nitrogen or noble gases), steam and carbon dioxide. The sweeper gas should not chemically react with any of the active compounds being removed from the plant matter. Alternatively, or additionally, a sweeper gas may be chosen to prevent thermal degradation of desired active compounds. An example would be: if a compound is prone to thermally decarboxylate then carbon dioxide could be chosen to shift the equilibrium in the desired product direction by providing an atmosphere which slows or stops such degradation. The pressure of the sweeper gas is adjusted using a pressure regulator (9) and the flow adjusted using a flow control valve (10). A check valve (11) is not required but is useful in isolating the gas supply from the thermal distillation process. The regulated sweeper gas then enters a well insulated housing or chamber (23), designed to minimize heat loss and minimize the formation of thermal gradients, where the TDR (13) is situated. If a heat transfer fluid is used then the housing is also designed to minimize fluid leakage.

In the arrangement shown the TDR (13) is located within the chamber (23) with an electrical heating element (15) and circulating fan (17), preferably, but not necessarily, internally mounted. The fan (17) circulates the fluid within the enclosure to produce uniform temperatures and to promote heat transfer between the circulating gas and the TDR (13). A vacuum oven, circulating oven or furnace may be adapted for this purpose. If a heat transfer fluid is used then an external heated tank and pump (not shown) are arranged to supply (34) temperature regulated fluid. The heating element and fan, and the external heating tank and pump, are different types of heat sources for the process. The temperature of the chamber (23) is carefully controlled to allow accurate temperature settings and thermal profile ramps to be utilized. A temperature or thermal profile ramp may be controlled using a proportional, proportional integral, or proportional integral derivative control system (16) to regulate the heat supplied to the chamber from the heating element (15) or the external heated tank and pump. The components (15), (16), and (17) form a heating loop. In such an arrangement the controller is programmed to heat the chamber at a certain heat rate (temperature profile ramp) and may be programmed to hold the temperature constant for a preset time hold period (i.e., a “soak” time) to enhance the separation of active components. Different heat rates and hold periods can be utilized within a single extraction process. If wet material is fed into the distillation system (25) the heating loop can be programmed to hold at a desired temperature to initially allow drying of the material to occur. An on-off control system may also be utilized for the heating loop.

The sweeper gas enters the insulated enclosure (23) and preferably passes through a preheater (12). The role of the preheater is to bring up the temperature of the sweeper gas to that within the oven prior to the sweeper gas entering the TDR (13). A long length of tubing, formed into a serpentine and located at the bottom enclosure (23) is suitable. Other shapes, and other configurations may also be used. For example, the tubing may have fins to increase the speed at which the sweeper gas is brought to the desired temperature. The sweeper gas flows through the manifold (3) and enters the lower plenum (14) of each channel. The upper side of the plenum (14) houses a fine mesh (not shown) which supports the material charged within the channel. The preheated sweeper gas flows through the channel (1) and contacts the plant material. The plant material is heated both through contact with the sweeper gas and through external heat transfer mechanisms through the wall (2) (FIG. 2) from the hot circulating gases which are heated via the heater (15) and circulated by the internal fan (17), or by the external heated tank and pump if a heat transfer fluid is used.

At certain stages during the heating ramp profile different active components are vaporized. The gases produced exit the end of the packed channel (1) and flow into fractionation header or collection manifold (5). The header allows the gas flow to be directed to a number of different condensers (19) where the desired product is collected. Manual valves, multi-port valves, and/or electronically actuated valves (18) can be used to direct (35) the gas flow among the various condensers. The gas flow from the collection manifold (5) is cooled by a condenser (19) to distill the extracted product, which is then collected in a collector (24). As the material is heated via the temperature ramp of the heating loop different gases are produced and the different the gases are redirected (36) by the valves (18) to different condensers (19) and are distilled for subsequent collection. The more volatile components will be produced initially, cooled by a first condenser (19A), and collected in the first collector (24A). At a temperature where the first “cut” has completed the valves (18) will then be actuated to end the flow to the first condenser (19A) and to direct the flow to a second condenser (19B) for collection of these components by a second collector (24B). The process is repeated until the upper temperature of the heating ramp has been achieved. The number of valves (18), condenser systems (19) and collectors (24) depends upon the number of active components within the plant material and the desired level of separation. FIG. 2 shows, as an example and not as a limitation, three valves (18A, 18B, 18C), three condensers (19A, 19B, 19C) and three collectors (24A, 24B, 24C). The condensers (19) can be any heat transfer device with a suitable cooling flow. The gases may also be cooled and collected by directly bubbling them through a liquid which is at least partially immiscible with the extracted active components from the plant material (not shown). A condenser and a collector, a bubbling water trap, or other type of bubbling trap, may be considered to be a product recovery system.

It may be desirable to enhance the extraction process by using a vacuum to promote vaporization and reduce the temperature at which the volatile components are removed from the plant material. This can prevent degradation of the active component from higher temperatures. A vacuum pump (20) can be used. Pressure sensors 22A-22C may be used to accurately measure the pressure at various points so that the operation of the vacuum pump may be controlled. This is particularly beneficial if one or more of the components to be extracted are thermally unstable.

The control system (16), in addition to controlling the heating process, preferably, but not necessarily, also uses pressure measurement information from the pressure sensors (22A-22C) to control the valve (10) and the vacuum pump (20), and preferably, but not necessarily, also uses temperature measure information from the temperature sensors (21A-21D), and an internal clock, to determine when to open and close various ones of the valves (18A-18C).

For example, the control system (16) may cause the heater (15) to heat the oven (23) to a first predetermined temperature, open a predetermined one of the valves (18) to allow a first extract to be condensed by a first condenser (19) and collected in a first collector (24), maintain that temperature for a first predetermined time, close that valve, cause the heater to heat the oven to a second predetermined temperature, open another one of the valves to allow a second extract to be condensed by a second condenser and collected in a second collector, maintain that temperature for a second predetermined time, close that valve, cause the heater to heat the oven to a third predetermined temperature, etc.

Alternatively, the valves (18) may be temperature-sensitive and open only when the temperature of the gas from the manifold (5) is within a predetermined temperature range.

The plant material typically contains a number of active components which may be removed (extracted) at different temperatures. For example, if Cannabis Sativa plant material is processed, some examples of extracted compounds and the boiling point and some properties of each are listed in Table 1 below. Tables 2 and 3 list, respectively, examples for Terpenoid Essential Oils, and examples for Flavonoid And Phytosterol Components.

TABLE 1
Cannabinoids

Δ-9-tetrahydrocannabinol (THC)
Boiling point: 157° C./314.6° Fahrenheit
Properties: Euphoriant, Analgesic, Antiinflammatory, Antioxidant,
Antiemetic
Cannabidiol(CBD)
Boiling point: 160-180° C./320-356° Fahrenheit
Properties: Anxiolytic, Analgesic, Antipsychotic, Antiinflammatory,
Antioxidant, Antispasmodic
Cannabinol (CBN)
Boiling point: 185° C./365° Fahrenheit
Properties: Oxidation, breakdown, product, Sedative, Antibiotic
Cannabichromene(CBC)
Boiling point: 220° C./428° Fahrenheit
Properties: Antiinflammatory, Antibiotic, Antifungal
Δ-8-tetrahydrocannabinol (Δ-8-THC)
Boiling point: 175-178° C./347-352.4° Fahrenheit
Properties: Resembles Δ-9-THC, Less psychoactive, More stable
Antiemetic
Tetrahydrocannabivarin(THCV)
Boiling point: <220° C./<428° Fahrenheit
Properties: Analgesic, Euphoriant

TABLE 2
Terpenoid Essential Oils

β-myrcene
Boiling point: 166-168° C./330.8-334.4 Fahrenheit
Properties: Analgesic. Antiinflammatory, Antibiotic, Antimutagenic
β-caryophyllene
Boiling point: 119° C./246.2° Fahrenheit
Properties: Antiinflammatory, Cytoprotective (gastric mucosa),
Antimalarial
D-limonene
Boiling point: 177° C./350.6° Fahrenheit
Properties: Potential Cannabinoid agonist Immune potentiator,
Antidepressant, Antimutagenic
Linalool
Boiling point: 198° C./388.4° Fahrenheit
Properties: Sedative, Antidepressant, Anxiolytic, Immune potentiator
Pulegone
Boiling point: 224° C./435.2° Fahrenheit
Properties: AChE inhibitor, Sedative, Antipyretic
1,8-cineole (eucalyptol)
Boiling point: 176° C./348.8° Fahrenheit
Properties: AChE inhibitor, Increases cerebral, blood flow, Stimulant,
Antibiotic, Antiviral, Antiinflammatory, Antinociceptive
α-pinene
Boiling point: 156° C./312.8° Fahrenheit
Properties: Antiinflammatory, Bronchodilator, Stimulant, Antibiotic,
Antineoplastic, AChE inhibitor
α-terpineol
Boiling point: 217-218° C./422.6-424.4° Fahrenheit
Properties: Sedative, Antibiotic, AChE inhibitor, Antioxidant,
Antimalarial
Terpineol-4-ol
Boiling point: 209° C./408.2° Fahrenheit
Properties: AChE inhibitor. Antibiotic
P-cymene
Boiling point: 177° C./350.6° Fahrenheit
Properties: Antibiotic, Anticandidal, AChE inhibitor
Borneol
Boiling point: 210° C./410° Fahrenheit
Properties: Antibiotic, Δ-3-carene 0.004% 168 Antiinflammatory
Δ-3-carene
Boiling point: 168° C./334.4° Fahrenheit
Properties: Antiinflammatory

TABLE 3
Flavonoid And Phytosterol Components

Apigenin
Boiling point: 178° C./352.4° Fahrenheit
Properties: Anxiolytic, Antiinflammatory, Estrogenic
Quercetin
Boiling point: 250° C./482° Fahrenheit
Properties: Antioxidant, Antimutagenic, Antiviral, Antineoplastic
CannflavinA
Boiling point: 182° C./359.6° Fahrenheit
Properties: COX inhibitor, LO inhibitor
β-sitosterol
Boiling point: 134° C./273.2° Fahrenheit
Properties: Antiinflammatory, 5-α-reductase, inhibitor

In one embodiment a parallel plate arrangement is utilized as the thermal distillation reactor (13). The channel (1) is formed by attaching two plates with a flanged side walls. The plates can be detached from one another to facilitate periodic cleaning of the channel. The TDF is mounted within a circulation oven (23) which uses temperature measurement devices (21A-21D) to accurately measure the temperature therein. A PID controller (16) controls or supplies the power to the heating element (15) to bring the temperature within the oven up to the desired temperature and to maintain it at that desired temperature. A controlled flow of nitrogen gas, such as from storage tank (8), flows through a length of tubing, for example, ¼″ internal diameter tubing, inside the oven (23) prior to entering the bottom of the TDR. The sweeper gas flows in an upwards direction (in the figure) towards the exit manifold (5), and then flows to the valves (18) and the condenser units (19). As the sweeper gas progresses through the channels (1) it contacts the plant material and sweeps the vaporizing active components towards the exit or collection manifold (5). In a preferred embodiment, water-cooled condensers (19) are selectively used to condense the vaporized fractions from the biomass. Other types of condensers may also be used. Each condenser operates at a temperature to allow condensation of the selected fraction to occur, but is sufficiently warm to maintain the product viscosity such that the fraction will still flow and collect in a collection vessel (24) located at the drain of the condenser. In the exemplary embodiment above, three collection vessels (24A, 24B, 24C) were used. In another exemplary embodiment, eight collection vessels are utilized to allow for eight different fractional cuts to be collected.

A number of different arrangements and different operational strategies may be used with the TDR. Some examples are given below. The list below is non exhaustive and is only to demonstrate the potential synergies and applications of the method and apparatus described herein.

### Example 1

A complete cannabis Sativa plant is harvested and macerated to produce an average particle size of 3 mm. The plant material is charged into a plate heat exchanger (13) with a channel dimension of 15 mm. A nitrogen flow with a Gas Hourly Space Velocity (GHSV) of 60 hr⁻¹ enters the bottom of the channel (1). The system is completely purged of oxygen. The heating system (15, 16, 17) ramps the temperature in the oven (23) from ambient to 100° C. preferably, but not necessarily, at 5° C. per minute. The oven temperature is held at 100° C. for around one hour or until the plant material is completely dried. The steam produced exits the top of the thermal distillation unit (13) and flows into a water cooled condenser, such as but not limited to the condenser (19C). The condenser cooling water temperature for the drying stage is around 20-25° C. The steam condenses within the condenser (19C) and flows into a corresponding water collection pot, such as but not limited to the collector (24C). Once drying has been completed the heating system (15,16,17) is programmed with the preferred ramp rates described in Table 4. A temperature ramp that is too steep may cause undesired temperature gradients within the plant material, whereas a temperature ramp that is too shallow unduly prolongs the time needed to extract the desired compounds.

TABLE 4
The Thermal Heating Loop Temperature Program

| | Temperature | Temperature | Ramp | Hold |
|---|---|---|---|---|
| Cut | Cut Start | Cut Finish | Rate | Time |
| Number | (° C.) | (° C.) | (° C./minute) | (Hrs) |
| 1 | 20 | 100 | 2 | 1 |
| 2 | 100 | 160 | 1 | 0.1 |
| 3 | 160 | 173 | 0.5 | 0.1 |
| 4 | 173 | 180 | 0.25 | 0.1 |
| 5 | 180 | 190 | 0.5 | 0.1 |
| 6 | 190 | 215 | 0.5 | 0.1 |
| 7 | 215 | 260 | 1 | 0.1 |

The cooling water flow within each condenser (19) is raised to 80° C. Condensing at this temperature sufficiently maintains the viscosity of the condensed products to allow the product to readily flow downwards from the selected condenser (19) and be collected within the relevant collection pot (24). Six cuts are achieved and product analysis reveals that each cut contains vastly different proportions of active components. In this example all the condensers (19) are at the same temperature. If desired, however, the condensers (19) may be operated at different temperatures, so as to minimize degradation or optimize the viscosity of the product being extracted. A summary of the major components collected within each fraction is Table 5.

TABLE 5
Major Components Within Each Fractional Cut.

| | Boiling Point | Fractional |
|---|---|---|
| Component | (° C.) | Cut Number |
| β-caryophyllene | 119 | 1 |
| β-sitosterol | 134 | 1 |
| a-pinene | 156 | 1 |
| Δ-9-tetrahydrocannabinol (THC) | 157 | 1 |
| β-myrcene | 167 | 2 |
| cannabidiol (CBD) | 170 | 2 |
| 1,8-cineole (eucalyptol) | 176 | 2 |
| Δ-8-tetrahydrocannabinol (Δ-8-THC) | 177 | 2 |
| d-limonene | 177 | 2 |
| p-cymene | 177 | 2 |
| Apigenin | 178 | 2 |
| cannflavin A | 182 | 3 |
| Cannabinol (CBN) | 185 | 3 |
| Linalool | 198 | 4 |
| terpineol-4-ol | 209 | 4 |
| cannabigerol (CBG) | 210 | 4 |
| tetrahydrocannabivarin (THCV) | 210 | 4 |
| a-terpineol | 218 | 5 |
| cannabichromene (CBC) | 220 | 5 |
| Pulegone | 224 | 5 |
| Quercetin | 250 | 6 |

### Example 2

The test described in Example 1 was repeated with the whole system being held under a vacuum and an operational pressure of 0.05 bar. The temperature of each cut is reduced by 50° C. The system operates in a similar manner and each cut contains essentially the same components. A higher content of the acid form of CBD and THC is found within the sample due to a lower rate of decarboxylation occurring at the lower temperatures.

### Example 3

The test described in Example 1 was repeated with carbon dioxide being used as the sweeper gas. The system operates in a similar manner and each cut contains essentially the same components. A higher content of the acid form of CBD and THC is found within the sample due to a lower rate of decarboxylation occurring due to the presence of carbon dioxide.

### Example 4

The test described in Example 1 was repeated. The plant material is air dried for 1 week prior to processing. The volume of water collected drying the initial ramp is found to be greatly reduced and this step can be omitted if the sample is air dried prior to processing. The system operates in a similar manner and each cut contains essentially the same components.

### Example 5

The test described in Example 1 was repeated with the condenser section being replaced by a bubbling water trap. In this arrangement the sweeper gas is directed towards the bottom of a vessel filled with water. The sweeper gas produces bubbles and flows in an upwards direction. The sweeper gas is cooled and active condensable products collect in the water phase. The trap fluid is cooled by an internal cooled coil. The product forms an immiscible layer. the bubbling water trap therefore functions as both the condenser and the collector. The immiscible layer is analysed and each cut is found to be similar to that noted within Example 1.

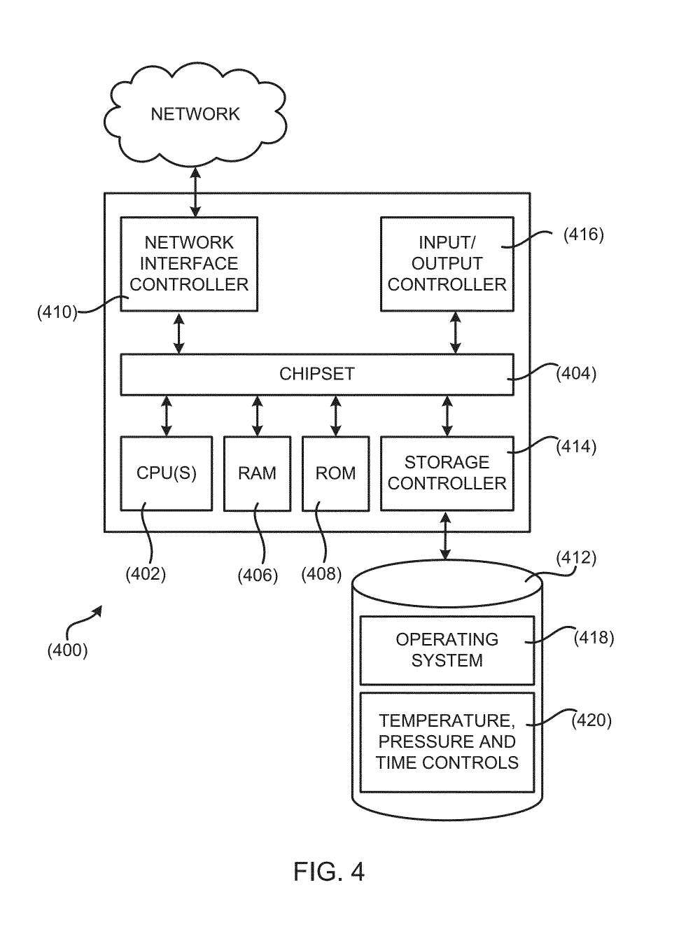

FIG. 4 shows an exemplary computer architecture for a computer (400) capable of executing the software components described herein for extracting desired products from plant material in the manner presented above. In operation, the computer (400) reads the instructions in module (420), reads the temperature and/or the pressure from one or more of the temperature or pressure sensors, applies the heating ramp profile, measures the time at a temperature, and changes the heating, pressure, vacuum, sweeper gas flow rate, and/or operation of the various valves to cause the macerated plant material to be heated and the gases resulting therefrom to be directed to various ones of the condensers and the collectors.

The computer architecture (400) shown in FIG. 4 illustrates a conventional server computer, workstation, desktop computer, laptop, PDA, electronic book reader, digital wireless phone, network appliance, set-top box, or other computing device, and may be utilized to execute any aspects of the software components presented herein.

The computer (400) includes a baseboard, or “motherboard,” which is a printed circuit board to which a multitude of components or devices may be connected by way of a system bus or other electrical communication paths. In one illustrative embodiment, one or more central processing units (“CPUs”) (402) operate in conjunction with a chipset (404). The CPUs (402) are standard programmable processors that perform arithmetic and logical operations necessary for the operation of the computer (400).

The CPUs (402) perform operations by transitioning from one discrete, physical state to the next through the manipulation of switching elements that differentiate between and change these states. Switching elements may generally include electronic circuits that maintain one of two binary states, such as flip-flops, and electronic circuits that provide an output state based on the logical combination of the states of one or more other switching elements, such as logic gates. These basic switching elements may be combined to create more complex logic circuits, including registers, adders-subtractors, arithmetic logic units, floating-point units, or the like.

The chipset (404) provides an interface between the CPUs (402) and the remainder of the components and devices on the baseboard. The chipset (404) may provide an interface to a random access memory (“RAM”) (406), used as the main memory in the computer (400). The chipset (404) may further provide an interface to a computer-readable storage medium such as a read-only memory (“ROM”) (408) or non-volatile RAM (“NVRAM”) for storing basic routines that help to start up the computer (400) and to transfer information between the various components and devices. The ROM (408) or NVRAM may also store other software components necessary for the operation of the computer (400) in accordance with the embodiments described herein.

According to various embodiments, the computer (400) may operate in a networked environment using logical connections to remote computing devices and computer systems through a network, such as a local-area network (“LAN”), a wide-area network (“WAN”), the Internet, or any other networking topology known in the art that connects the computer (400) to remote computers. The chipset (404) includes functionality for providing network connectivity through a network interface controller (“NIC”) (410), such as a gigabit Ethernet adapter. For example, the NIC (410) may be capable of connecting the computer (400) to other computing devices. It should be appreciated that multiple NICs (410) may be present in the computer (400), connecting the computer to other types of networks and remote computer systems.

The computer (400) may be connected to a mass storage device (412) that provides non-volatile storage for the computer. The mass storage device (412) may store system programs, application programs, other program modules, and data, which have been described in greater detail herein. The mass storage device (412) may be connected to the computer (400) through a storage controller (414) connected to the chipset (404). The mass storage device (412) may consist of one or more physical storage units. The storage controller (414) may interface with the physical storage units through a serial attached SCSI (“SAS”) interface, a serial advanced technology attachment (“SATA”) interface, a FIBRE CHANNEL (“FC”) interface, or other standard interface for physically connecting and transferring data between computers and physical storage devices.

The computer (400) may store data on the mass storage device (412) by transforming the physical state of the physical storage units to reflect the information being stored. The specific transformation of physical state may depend on various factors, in different implementations of this description. Examples of such factors may include, but are not limited to, the technology used to implement the physical storage units, whether the mass storage device (412) is characterized as primary or secondary storage, or the like. For example, the computer (400) may store information to the mass storage device (412) by issuing instructions through the storage controller (414) to alter the magnetic characteristics of a particular location within a magnetic disk drive unit, the reflective or refractive characteristics of a particular location in an optical storage unit, or the electrical characteristics of a particular capacitor, transistor, or other discrete component in a solid-state storage unit. Other transformations of physical media are possible without departing from the scope and spirit of the present description, with the foregoing examples provided only to facilitate this description. The computer (400) may further read information from the mass storage device (412) by detecting the physical states or characteristics of one or more particular locations within the physical storage units.

In addition to the mass storage device (412) described above, the computer (400) might have access to other computer-readable media to store and retrieve information, such as program modules, data structures, or other data. It should be appreciated by those skilled in the art that computer-readable media can be any available media that may be accessed by the computer (400), including computer-readable storage media and communications media. Communications media includes transitory signals. Computer-readable storage media includes volatile and non-volatile, removable and non-removable storage media implemented in any method or technology. For example, computer-readable storage media includes, but is not limited to, RAM, ROM, erasable programmable ROM (“EPROM”), electrically-erasable programmable ROM (“EEPROM”), flash memory or other solid-state memory technology, compact disc ROM (“CD-ROM”), digital versatile disk (“DVD”), high definition DVD (“HD-DVD”), BLU-RAY, or other optical storage, magnetic cassettes, magnetic tape, magnetic disk storage or other magnetic storage devices, or any other medium that can be used to store the desired information. Computer-readable storage media does not include transitory signals nor any construction prohibited by statute.

The mass storage device (412) may store an operating system (418) utilized to control the operation of the computer (400). According to one embodiment, the operating system comprises the LINUX operating system. According to another embodiment, the operating system comprises the WINDOWS® SERVER operating system from MICROSOFT Corporation of Redmond, Wash. According to further embodiments, the operating system may comprise the UNIX or SOLARIS operating systems. It should be appreciated that other operating systems may also be utilized. The mass storage device (412) may store other system or application programs and data utilized by the computer (400), such as the temperature, pressure, and time controls module (420).

In one embodiment, the mass storage device (412) or other computer-readable storage media may be encoded with computer-executable instructions that, when loaded into the computer (400), transform the computer from a general-purpose computing system into a special-purpose computer capable of implementing the embodiments described herein. These computer-executable instructions transform the computer (400) by specifying how the CPUs (402) transition between states, as described above. According to one embodiment, the computer (400) has access to computer-readable storage media storing computer-executable instructions that, when executed by the computer, perform the various routines and operations described herein.

The computer (400) may also include an input/output controller (416) for receiving and processing input from a number of input devices, such as a keyboard, a mouse, a touchpad, a touch screen, an electronic stylus, or other type of input device. Similarly, the input/output controller (416) may provide output to a display device, such as a computer monitor, a flat-panel display, a digital projector, a printer, a plotter, or other type of output device. It will be appreciated that the computer (400) may not include all of the components shown in FIG. 4, may include other components that are not explicitly shown in FIG. 4, or may utilize an architecture completely different than that shown in FIG. 4.

Based on the foregoing, it should be appreciated that an apparatus and a method for non-solvent-based extraction of compounds from plant material has been disclosed herein. Although the subject matter presented herein has been described in language specific to systems, methodological acts, mechanical and physical operations and/or configurations, and manufacturing processes, it is to be understood that the invention disclosed herein is not necessarily limited to the specific features, configurations, or components described herein. Rather, the specific features, configurations and components are disclosed as example forms. Further, all of the various features, configurations, and components need not be embodied in a single item to gain the benefits of other features, configurations, and components.

The subject matter described herein is provided by way of illustration for the purposes of teaching, suggesting, and describing, and not limiting. Alternatives to the illustrated embodiment are contemplated, described herein, and set forth in the claims. Various modifications and changes may be made to the subject matter described herein without following the example embodiments and applications illustrated and described, and without departing from the true spirit and scope of the present invention.

Also, some benefits and features may be obtained independently of other features and benefits. For example, although an apparatus and method for extracting numerous different compounds have been described, the apparatus and method may be used to extract five, four, three, or two compounds, or even a single compound. Furthermore, some of the benefits and features described herein may be obtained without use of all of the components and/or steps described herein. In addition, the features, functions, and advantages that have been discussed can be achieved independently in various embodiments of the present disclosure or may be combined in yet other embodiments.

1. An apparatus to thermally fractionate and recover compounds contained within plant material, the apparatus comprising:
a sweeper gas preheater to preheat a received sweeper gas and provide a preheated sweeper gas;
a distribution manifold to receive and distribute the preheated sweeper gas along a plurality of paths;
a plurality of channels, each channel corresponding to one of the paths from the distribution manifold to receive the preheated sweeper gas, each channel to receive macerated plant material, and to extract a gaseous product from the macerated plant material;
a product collection manifold to receive and combine the gaseous product from the plurality of channels;
a product recovery system to receive the combined gaseous product from the product collection manifold;
a heating device to heat either a liquid or a gas to a predetermined temperature, the plurality of channels being bathed in and heated by the liquid or the gas from the heating device; and
an insulated container enclosing the distribution manifold, the plurality of channels, the product collection manifold, and the product recovery system.

2. The apparatus of claim 1 where a channel of the plurality of channels comprises a sealed arrangement of parallel plates.

3. The apparatus of claim 1 where a channel of the plurality of channels comprises the inner annulus of a tube or pipe.

4. The apparatus of claim 1 where a channel of the plurality of channels has a characteristic inner diameter in the range 0.15 to 6″.

5. The apparatus of claim 1 wherein the heating device comprises an electrically powered heating element, a circulation fan, and a temperature measurement device, the heating device being within the enclosure, the heating device controlling power supplied to the heating element based upon an output of the temperature measurement device.

6. The apparatus of claim 1 wherein:
the product recovery system comprises a plurality of combinations connected in parallel, each combination comprising a valve and a condenser connected in series, with a collector connected to the condenser; and
a valve of a combination passes the gaseous product from the collection manifold to a respective condenser when a temperature of the gaseous product is within a predetermined range.

7. The apparatus of claim 1 wherein each condenser has an input connected to a corresponding valve and an output; and
further comprising a vacuum pump connected to the outputs of the condensers.

8. The apparatus of claim 1 wherein each channel of the plurality of channels is constructed to allow the channel to be opened to provide for cleaning of the channel.

9. The apparatus of claim 1 wherein the sweeper gas preheater is external to the insulated container.

10. The apparatus of claim 1 and further comprising a control system to control at least one of the sweeper gas preheater, the heating device, valves in the product recovery system, or a vacuum pump.

11. A method to thermally fractionate and recover compounds from plant based material, the method comprising:
loading macerated plant material into a plurality of channels;
introducing a sweeper gas flow along the length of each channel;
externally heating each channel in accordance with a temperature controlled ramp;
directing gas exiting the channels in a first temperature range to a first condenser; and
directing the gas exiting the channels in a second temperature range to a second condenser;
whereby the first condenser provides a first compound and the second condenser provides a second compound.

12. The method of claim 11 and further comprising vibrating the channels during at least a portion of a time that the macerated plant material is being loaded into the channels to assist in loading the macerated plant material evenly throughout the channels.

13. The method of claim 11 wherein the sweeper gas is inert or does not react with or degrade any of the components released from the plant material.

14. The method of claim 11 wherein the sweeper gas minimizes or at least reduces thermal degradation of at least one of the first compound or the second compound.

15. The method of claim 11 and further comprising directing the gas exiting the channels in a third temperature range to a third condenser.

16. The method of claim 11 and further comprising providing a cooling liquid to at least one of the first condenser or the second condenser to facilitate the collection of the product.

17. The method of claim 11 and further comprising bubbling the gas exiting the channels through a cooling liquid to facilitate the collection of the product.

18. The method of claim 11 wherein the macerated plant material is wet, and further comprising externally heating each channel in accordance with a drying cycle prior to heating each channel in accordance with the temperature controlled ramp.

19. The method of claim 11 and further comprising macerating plant material to provide the macerated plant material.

20. An apparatus to thermally fractionate and recover compounds contained within plant material, the apparatus comprising:
a pressure regulator to control the pressure of a sweeper gas to provide a controlled-pressure sweeper gas, the sweeper gas being at least one of an inert gas, a gas which does not react with components in the plant material, or a gas which does not degrade components in the plant material;
a valve connected to the pressure regulator to control the flow of the sweeper gas;
a sweeper gas preheater to preheat the controlled-flow sweeper gas and provide a preheated sweeper gas;
a distribution manifold to receive and distribute the preheated sweeper gas along a plurality of paths;
a plurality of channels, each channel corresponding to one of the paths from the distribution manifold to receive the preheated sweeper gas, each channel to receive macerated plant material, and to extract a gaseous product from the macerated plant material;
a product collection manifold to receive and combine the gaseous product from the plurality of channels;
a plurality of valves connected to the product collection manifold to selectively direct the gaseous product among a corresponding plurality of product recovery systems;
each product recovery system of the plurality of product recovery systems to selectively receive the gaseous product and provide an extracted product;
a heating device to heat either a liquid or a gas to a predetermined temperature, the plurality of channels being bathed in and heated by the liquid or the gas from the heating device;
an insulated container enclosing the distribution manifold, the plurality of channels, the product collection manifold, and the product recovery system;
at least one temperature sensor to measure the temperature inside the insulated container;
a vacuum pump to selectively apply a vacuum to the product recovery systems;
at least one pressure sensor to measure the pressure inside the insulated container; and
a controller, responsive to the temperature and to the pressure, and to predetermined instructions, to control the operation of the valve connected to the pressure regulator, the sweeper gas preheater, the plurality of valves, the heating device, and the vacuum pump.
